(19) United States
(12) Patent Application Publication
(10) Pub. No.: US 2020/0377862 A1
(43) Pub. Date: Dec. 3, 2020

(54) METHOD FOR PREPARING CANCER STEMNESS CELL LINE THROUGH METABOLIC STRESS, AND CANCER CELL PREPARED THROUGH SAME

(71) Applicant: YOUNSEI UNIVERSITY INDUSTRY FOUNDATION, Seoul (KR)

(72) Inventors: Jaemoon YANG, Seoul (KR); Jin-Suck SUH, Seoul (KR); Minhee KU, Seoul (KR)

(73) Assignee: YOUNSEI UNIVERSITY INDUSTRY FOUNDATION, Seoul (KR)

(21) Appl. No.: 16/606,795

(22) PCT Filed: Apr. 20, 2018
(86) PCT No.: PCT/KR2018/004611

§ 371 Date: Oct. 21, 2019

(30) Foreign Application Priority Data
Apr. 21, 2017 (KR) 10-2017-0051545

(51) Int. Cl. C12N 5/095

(52) U.S. Cl. CPC C12N 5/0695; C12N 2509/00

(57) ABSTRACT

The present disclosure relates to a cancer cell line having a stem cellularity and a method for producing the same, and more specifically, to inducing cancer cells to cancer stem cells by applying metabolic stress. More specifically, the present disclosures relates to the production and establishment of the cancer stem cell by repeating culturing in a glucose-deficient medium. The main features of the cancer stem cell produced by the method are that, in a glucose-deficient environment, the resistance to apoptosis is exhibited high and the resistance to anticancer drugs is highly exhibited high.

### TECHNICAL FIELD

The present disclosure relates to a method for preparing a cancer stem cell and a cancer cell line prepared thereby, and more particularly, to a method for preparing a cancer cell line having stem cell properties through metabolic stress and a cancer cell line prepared thereby.

### BACKGROUND ART

The cells identified to have stem cell-like characteristics in tumors have raised the question of whether tumors originate from these cells, but much evidence has been reported that cancer stem cells are involved in tumor development. The interest in cancer stem cells may be attributed to the failure of chemotherapy to target and treat tumors effectively and the recurrence of tumors. Many cytotoxic anticancer drugs usually target rapidly proliferating cells, so the responses to drugs may actually be responses to transitional amplification cells, and cancer stem cells with slowly proliferating characteristics are assumed to survive cytotoxic chemotherapy. Basal cell phenotype breast cancer is regarded as originating from the earliest mammary progenitor cells in an early stage of the differentiation process and is known to have a poor prognosis and to be resistant to conventional chemotherapy. It is a good example supporting that the reason for the failure of chemotherapy is because the targeted treatment for cancer stem cells has not been performed.

In the case of identifying diseases and optionally targeting drugs, cancer stem cells can have an impact on cancer treatment, including prevention of metastasis and new treatment strategies. Normal somatic stem cells are resistant to chemotherapy. They form various pumps to release DNA repair proteins and drugs. Normal somatic stem cells have slow cell replacement rates (chemotherapeutic agents naturally target cells that self-replicate fast). Cancer stem cells developed from normal stem cells can produce proteins that increase their resistance to chemotherapy. Surviving cancer stem cells allow tumors to regenerate as a cause of recurrence. Selective targeting of cancer stem cells is an aggressive treatment method that not only prevents metastasis and recurrence, but also does not excise tumors.

Upon reviewing the method for isolating these cancer stem cells, as cancer stem cells are mostly reported in human tumors, strategies to identify cells that are similar to normal stem cells are used across studies. These procedures include fluorescence-activated cell sorting (FACS), functional approaches including antibodies directed at cell-surface markers and side population assay, or Aldefluor assay. Various results regarding cancer stem cell is then used to assess tumor development capacity in immune-deficient mice when multiple drugs are administered. This in vivo assay is called a limiting dilution assay. The tumor cell subsets that can initiate tumor development at low cell numbers are further tested for self-renewal capacity in serial tumor studies. Cancer stem cells can also be identified by multidrug resistance (MDR) through efflux of incorporated Hoechst dyes via ATP-binding cassette (ABC) transporters. Another approach is sphere-forming assays. Many normal stem cells such as hematopoietic or stem cells from tissues, under special culture conditions, form three-dimensional spheres that can differentiate. As with normal stem cells, the cancer stem cells isolated from brain or prostate tumors also have the ability to form anchor-independent spheres.

However, there are many methods for isolating cancer stem cells that play an important role in the study of anticancer drugs, but the method for producing cancer stem cells themselves has been rarely studied such that the need for this is increasing.

### SUMMARY OF INVENTION

### Technical Problem

A technical task of the present disclosure is to provide a method for producing a cancer cell line having the characteristics of stem cells and a stem cell cancer cell line produced thereby, thereby broadening the understanding of cancer stem cells and also developing anticancer drugs targeting cancer stem cells.

Technical tasks to be achieved by the present disclosure are not limited to the aforementioned technical tasks, and other technical tasks, which are not mentioned herein, will be clearly understood from the following description by those having ordinary skill in the technical field to which the present disclosure pertains.

### Solution to Problem

An exemplary embodiment of the present disclosure provides a method for producing a cancer stem cell through metabolic stress and a cancer cell line produced by the method.

The cancer stem cell according to one aspect of the present disclosure may be derived from cancer cells and induced after being cultured in a chronic glucose-deficient medium, and the cancer stem cell may have a mitochondrial remodeling.

The cancer cell may be a breast cancer cell.

The cancer cell may be a gastric cancer cell.

In the mitochondrial remodeling, the mitochondrial fusion and cleavage are repeated, but the fusion is relatively predominant, and the average length of the mitochondria is increased.

In relation to a biomarker, the cancer stem cell shows the characteristics that the expression of stem cell markers CD44, ESA and SSEA-3 increases as compared to that of parent cells before induction, but the expression of Oct4 remains unchanged.

The cancer stem cell may include at least one of the following characteristics in relation to mitochondria:

(1) Glucose absorption rate is lower than parent cells before induction.

(2) The ratio of nucleus DNA (nDNA) to mitochondrial DNA (mtDNA) is higher than that of parent cells before induction, and mitochondria have a long shape by fusion.

(3) The concentration of cytosolic NAD+/NADH involved in cellular energy metabolism is higher than that of parent cells before induction.

(4) The expression of pCREB and PGC-la involved in cAMP-PKA signal transduction is higher than that of parent cells before induction in the cell nucleus and cell substrate, respectively.

(5) OCR (Oxygen Consumption Rate) increases.

The cancer stem cell may include at least one of the following characteristics.

(1) The ability to resist apoptosis in glucose-deficient environment is higher than that of parent cells before induction.

(2) Anticancer drug resistance is higher than parent cells before induction.

A method for producing cancer stem cell according to another aspect of the present disclosure includes:

culturing the isolated cancer cells in a nutrient medium;

removing the nutrient medium and adding a glucose-deficient nutrient medium;

maintaining the culture for at least 3 days in the glucose-deficient medium; and

establishing a cancer cell line by repeating a process of obtaining surviving cancer cells after the maintaining step and maintaining them in a glucose deficient medium again.

The method may further include pulverizing the cancer tumor tissues obtained from cancer patients and decomposing the matrix to isolate cancer cells before culturing the cancer cells in the nutrient medium.

The cancer cell may be a breast cancer cell.

The cancer cell may be a gastric cancer cell.

The maintaining of the culture may include maintaining until the time when the number of cancer cells falls below 20% of the number of cancer cells before the glucose-deficient nutrient medium is added.

The maintaining of the culture may include maintaining until the time when the number of cancer cells falls below 10% of the number of cancer cells before the glucose-deficient nutrient medium is added.

The repeating process may be performed seven or more times.

### Advantageous Effects of Invention

According to one embodiment of the present disclosure, the cancer stem cell prepared through metabolic stress has increased resistance to apoptosis. According to another embodiment of the present disclosure, the cancer stem cell prepared through metabolic stress has increased resistance to anticancer drugs as compared to the conventional cancer cells.

It should be understood that the effects of the present disclosure are not limited to those described above, and the present disclosure includes all effects that can be deduced from the detailed description of the present disclosure or the configurations of the invention described in the claims.

### BRIEF DESCRIPTION OF DRAWINGS

FIG. 1 shows a process of inducing and culturing cancer cell lines in a chronic glucose-deficient culture medium.

FIG. 2 shows (a) CD44 and ESA expression and (b) SSEA-3 and Oct4 expression with a flow cytometry in cancer cells before induction and induced cancer cells in order to measure the stem cell characteristics of cancer cells induced through a chronic glucose-deficient medium.

FIG. 3 shows phase contrast microscopy images of sphere formations of cancer cells before induction and induced cancer cells in order to measure the stem cell characteristics of cancer cells induced through a chronic glucose deficient medium.

FIG. 4 shows the number of sphere formations of cancer cells before induction and induced cancer cells in order to measure the stem cell characteristics of cancer cells induced through a chronic glucose deficient medium.

FIG. 5 shows the results of treatment of induced cancer cells with anticancer drugs (a) Paclitaxel, (b) Cisplatin in order to measure the anticancer drug resistance of cancer cells induced through a chronic glucose deficient medium.

FIG. 6 shows an image taken with a confocal microscope after staining (a) cancer cells before induction and (b) induced cancer cells with Mitotracker Red CMXRos dye. Blue indicates nucleus staining with Hoechest33342 dye.

FIG. 7 is a confocal microscope image of mitochondrial cell membrane potential by staining mitochondria in cancer cells before induction and induced cancer cells with JC-1 in order to measure mitochondrial changes in cancer cells induced through a chronic glucose-deficient medium. J-aggregates show red fluorescence as the monomer JC-1 (green) becomes aggregates when cell membrane potential increases as cell respiration progresses.

FIG. 8 shows a graph of J-aggregates fluorescence intensity of images taken with a confocal microscope for mitochondrial cell membrane potential by staining mitochondria in cancer cells before induction and induced cancer cells with JC-1 in order to measure mitochondrial changes in cancer cells induced through a chronic glucose-deficient medium.

FIG. 9 shows the measured values of oxygen consumption rate (OCR) of cancer cells (solid lines) induced through a chronic glucose deficient medium and cancer cells before induction (dotted lines) for oligomycin, FCCP, and rotenone/antimycin A.

FIG. 10 shows the correlation between the extracellular acidification rate (ECAR) and the oxygen consumption rate (OCR) of cancer cells before induction (thin lines) and cancer cells (bold lines) induced through a chronic glucose-deficient medium.

### DESCRIPTION OF EMBODIMENTS

Hereinafter, embodiments of the present disclosure will be described in detail so as to be easily carried out by those having ordinary skill in the technical field to which the present disclosure pertains. The present disclosure may, however, be embodied in various different forms and is not limited to the embodiments set forth herein.

### Example 1: Construction and Culture of Chronic Metabolic Stress Induced Cell Line (MDA-MB-231. MCF7)

Breast cancer cells (MCF7 and MDA-MB-231) were subcultured to form cancer stem cells in the metabolic stress according to the present disclosure. The breast cancer cell line was purchased from Korea Cell Line Bank (KCLB, Korea Cell line Bank, Seoul, Korea). The cell line was incubated in a 37° C., 5% CO₂ incubator in RPMI1640 (GIBCO Invitrogen Carlsbad, Calif., USA) nutrient medium containing 10% fetal bovine serum (FBS; GIBCO Invitrogen Carlsbad, Calif., USA) according to the protocol presented by the Korea Cell Line Bank.

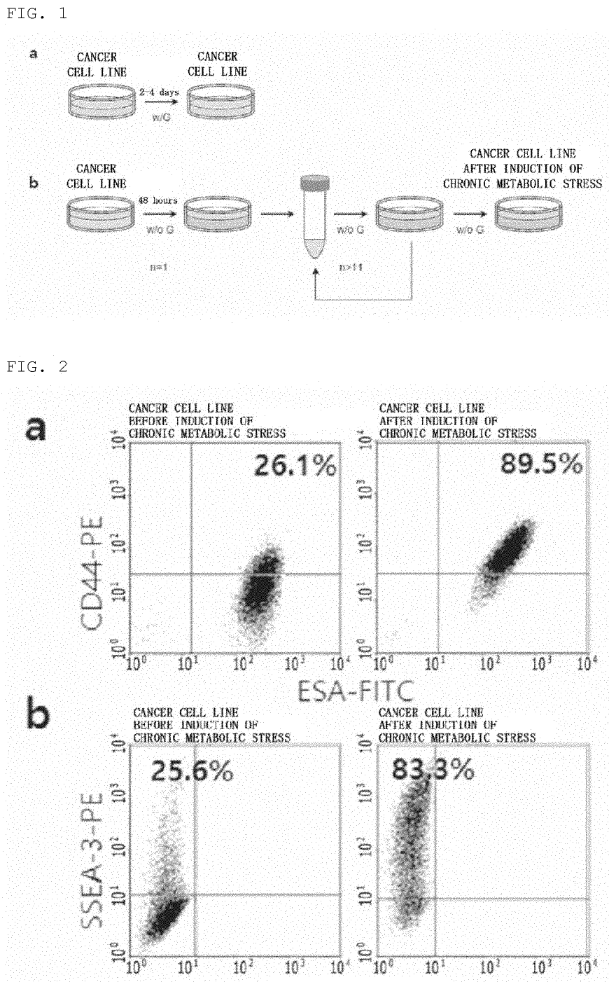

For the induction of chronic breast cancer, the culture was continuously performed by removing the cancer cells cultured in the nutrient medium (including glucose) from the existing nutrient medium, and replacing the existing nutrient medium with glucose-deficient medium RPMI1640, no glucose (GIBCO Invitrogen Carlsbad, Calif., USA). As shown in (a) of FIG. 1, the control group was cultured on the same day under a nutrient medium condition containing nutrient medium sugar including glucose. For the experimental group (cancer stem cell according to the present disclosure), a culture process performed in the glucose-deficient medium was repeated by re-obtaining the cells that survived in cancer cells cultured in a glucose-deficient medium as shown in (b) of FIG. 1.

### Example 2: Cell Viability and Proliferation

Cells attached and grown in a medium were suspended in a single cell unit using TrypLE™ Express (GIBCO Invitrogen Carlsbad, Calif., USA). For cell proliferation, 1×10⁴ cells were seeded in 96-well culture plates, and the viability of the cells was analyzed by colorimetric assay based on cell degeneration of 3-(4,5-dimethylthiazoly-2)-2,5-diphenyltetrazolium bromide (MTT) (Cell Proliferation Kit I, Roche, Germany) for metabolically active cells. In cell viability experiments, cells were seeded in 96-microwell plates (Thermo Fisher Scientific, Waltham, Mass., USA) and incubated for 24 hours at 37° C., 5% CO₂. After incubation, 10 μl of a yellow MTT solution was treated and incubated for an additional 4 hours, at which time purple formazan crystals formed by mitochondrial activity was melted with a 10% sodium dodecyl sulfate solution containing 100 μl of 0.01 M HCl. The absorbance of the solution was measured at 584 nm and 650 nm, respectively, using a microplate spectrometer (Epoch™, BioTek, VT, USA), and the absorbance value at the reference value of 650 nm was subtracted from the absorbance value at 584 nm. The cell viability was determined by the absorption intensity shown in the experimental group compared to the control group (mean±standard deviation (n=3)).

### Test Example 1: Cancer Stem Cellularity (Sternness) of Chronic Breast Cancer Cells

For FACS analysis, the chronic metabolic stressed breast cancer cells and control breast cancer cells 5×105 cells were separated into single cells, followed by staining at 4° C. for 30 minutes using an antibody. After cell immobilization, an analysis was conducted using flow cytometry (BD Facscalibur, BD Bioscience, CA, USA). The FACS analysis was performed using CD44, SSEA-3, ESA, and OCT4 corresponding to cancer stem cell markers, and the results are shown in (a) of FIG. 2. In addition, each of the cells was cultured for 14 days with a sphere formation medium in an Ultra-Low Attachment 6-well plate (Thermo Fisher Scientific, Waltham, Mass., USA) for sphere formation experiments.

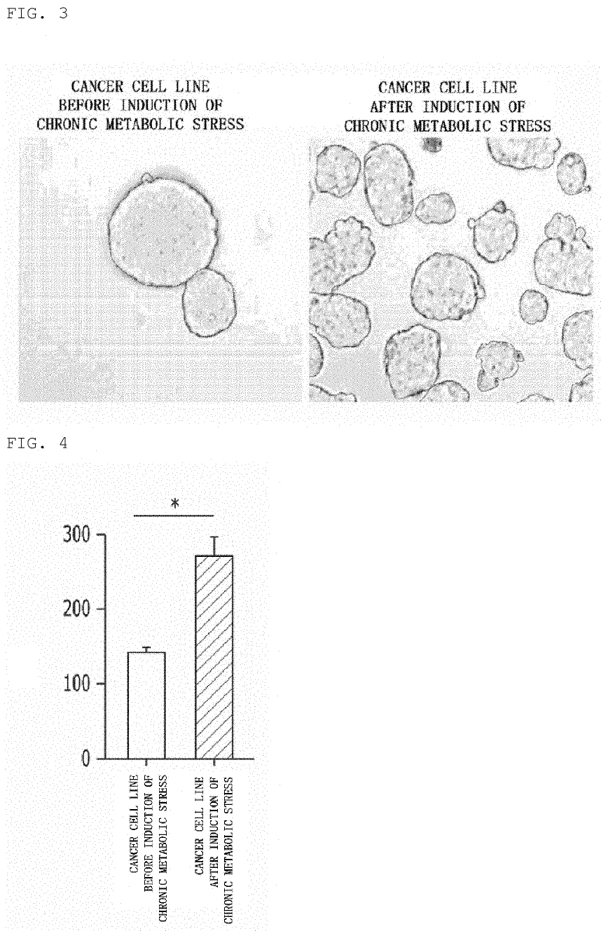

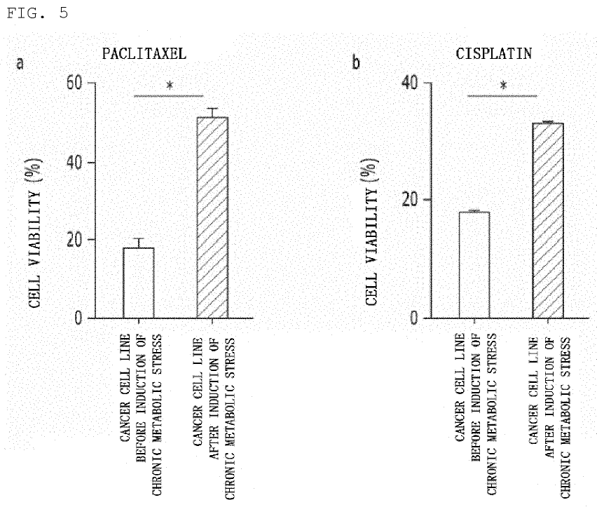

Each of the cultured cells was observed under a microscope to count the number of spheres formed in each cell, and the results are shown in FIGS. 3 and 4.

As shown in FIGS. 2, 3 and 4, it was found that the expression of SSEA-3, CD44 and ESA, which are markers of cancer stem cells, increased significantly as compared to the control (parental, p) in chronic breast cancer cell lines (chronic, c) cultured in a glucose-free environment, and the number of sphere formation cell populations also increased by three or more times.

The results suggest that chronic breast cancer cell lines cultured in a glucose-deficient environment obtain cancer stem cellularity (sternness).

### Test Example 2: Anticancer Drug Resistance in Chronic Breast Cancer Cells

In order to confirm the anticancer drug resistance of chronic breast cancer cells, each of chronic stressed breast cancer cells (Chronic) and breast cancer cells in control groups (Parental) were treated with Paclitaxel and Cisplatin, which are commonly used anticancer drugs in breast cancer. After the same measurement as the method for evaluating the viability was performed, the results are shown in (a) and (b) of FIG. 3.

As shown in (a) and (b) of FIG. 5, it was found that when treated with Paclitaxel and Cisplatin, the viability of chronic breast cancer cells (Chronic) was increased about 2 or 3 times as compared to breast cancer cells in control groups (Parental).

The results suggest that anticancer drug resistance was obtained in chronic breast cancer cell lines exposed to a glucose-deficient environment.

### Test Example 3: Changes in Mitochondrial Activity in Chronic Metabolic Stress Breast Cancer Cells

In order to confirm the change in the activity of mitochondria, which is a major organ of energy metabolism, in chronic MCF7 (chronic), mitochondrial morphology was confirmed and capacity was measured.

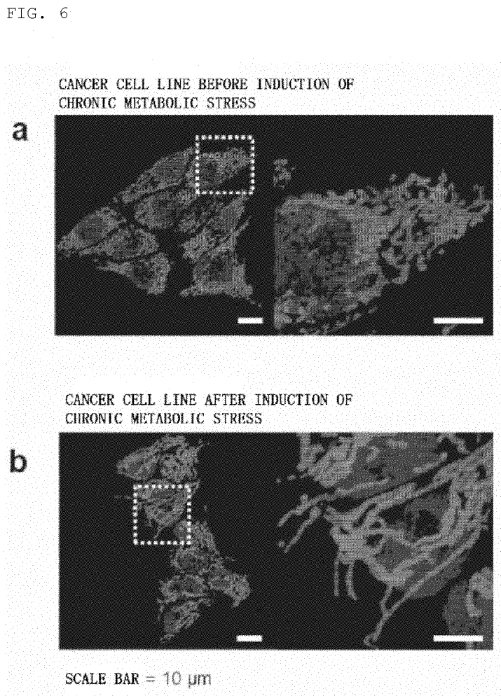

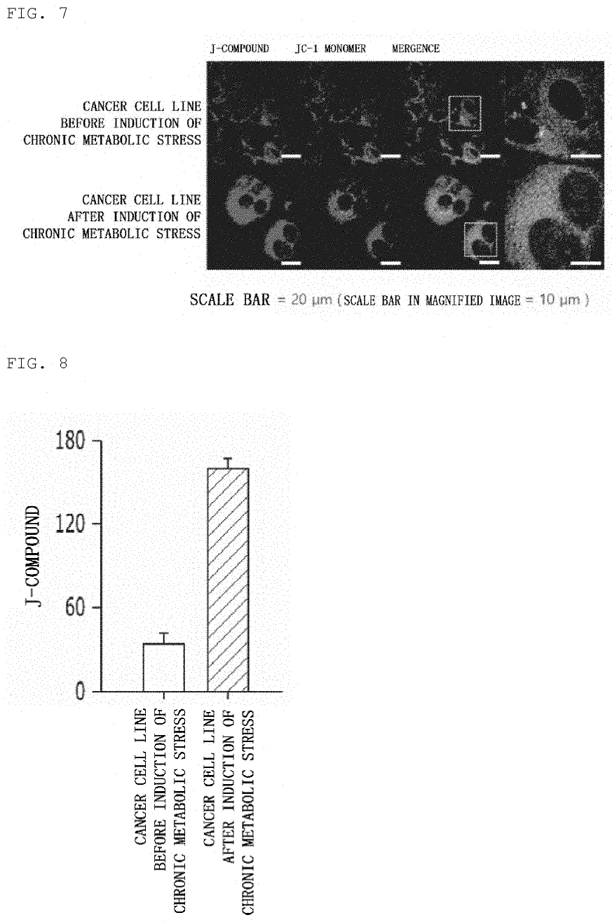

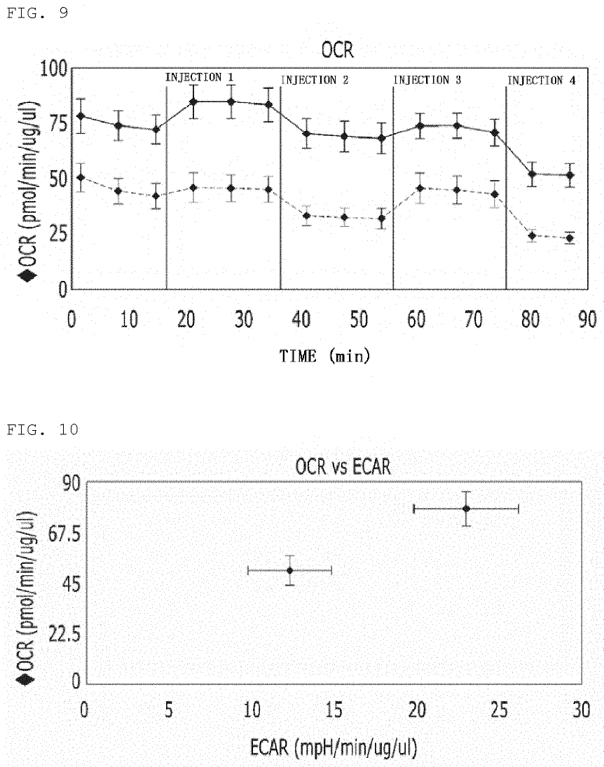

For measuring mitochondrial membrane potential and oxidative phosphorylation of living cells, MitoTracker® Red CMXRos and JC-1 Dye with 150 nM fluorescence bound according to the protocol provided by the manufacturer (Invitrogen) were treated. After 45 minutes of reaction, immobilization using 4% paraformaldehyde was performed, Hoechst33342 was treated for 30 minutes for nuclear staining, followed by observation under a confocal microscopy (LSM700, Carl Zeiss, Jena, Germany). The results are shown in FIGS. 6 and 7. The result of quantifying the observed fluorescence intensity is shown in FIG. 8. In addition, for the measurement of basal oxygen consumption and maximum respiration volume, each of the chronic breast cancer cell line (MCF7 (chronic)) and breast cancer cell line in control groups (MCF7 (parental)) was cultured by 2×10⁴ cells for 2 days in a cell culture plate dedicated for Seahorse XFe24 Extracellular Flux Analyzer (Seahorce Bioscience Inc., North Billerica, Mass., USA). After 2 days, oligomycin, carbonyl cyanide-4-(trifluoromethoxy)phenylhydrazone (FCCP), rotenone and antimycin drugs were mixed and treated, and the results measured through the XFe24 Extracellular Flux Analyzer are shown in FIGS. 9 and 10.

As shown in FIGS. 6, 7 and 8, the mitochondrial membrane potential in the chronic metabolic stress cell line MCF7 (chronic) was confirmed. According to the Mitotracker and JC-1 dye characteristics, which can predict the distribution and morphology of mitochondria, it was confirmed that the expression of J-aggregate with red fluorescence formed by the change of mitochondrial membrane potential increased by the effect of cellular respiration. Moreover, it was found that mitochondria is fused to have long shapes.

In addition, as shown in FIGS. 9 and 10, in the chronic metabolic stress cell line MCF7 (chronic), the oxidative phosphorylation (oxidative phosphorylation) was found to be about 2 times higher, and the oxidative phosphorylation capacity showed a lower reduction rate than the control cells (MCF7 (parental)) due to the drug.

The description of the present disclosure described above are for illustrative purposes, and those having ordinary skill in the technical field to which the present disclosure pertains may understand that the exemplary embodiments may be implemented in other specific embodiments without changing the technical spirit or essential features of the present disclosure. Accordingly, the aforementioned exemplary embodiments are only examples in every aspect and thus, are to be understood not to be limitative. For example, each component described as a single type may be implemented in a distributed manner, and similarly, components described as distributed may also be implemented in a combined form.

### Embodiments

Hereinafter, the present disclosure will be described with reference to the accompanying drawings. The present disclosure, however, may be implemented in various different forms, and thus it is not limited to embodiments explained herein. In addition, in the drawings, some portions not related to the description will be omitted in order to clearly describe the present disclosure, and similar reference numerals are given to similar parts throughout the specification.

Throughout the specification, when a portion is referred to as being “connected” (accessed, contacted, or coupled) to other portion, it may include a case in which the portion is “directly connected” to the other portion as well, and a case in which the portion is “indirectly connected” to the other portion with another member interposed therebetween. Also, when a portion is referred to as “including” a component, it may mean that another component is further included and is not to be excluded unless specifically stated otherwise.

Terms used herein are for the purpose of describing only specific embodiments and are not intended to be limiting of the present disclosure. Unless the context clearly dictates otherwise, the singular form includes the plural form. In this present specification, the terms “comprising,” “having,” or the like are used to specify that a feature, a number, a step, an operation, a component, an element, or a combination thereof described herein exists, and they do not preclude the presence or addition of one or more other features, numbers, steps, operations, components, elements, or combinations thereof.

Hereinafter, embodiments of the present disclosure will be described in detail with reference to the accompanying drawings.

The term “cancer stem cells” refers to cancer cells having the ability to self-renew, proliferate, and multi-differentiate by forming a microenvironment in which blood vessels, mesenchymal cells, and various types of cancer cells are gathered. The cancer stem cells can proliferate at a slow rate, unlike ordinary cancer cells, under a normal tumor growth condition, or can be kept in a dormant state, and thus can have resistance to anticancer drugs. Specifically, expression of transcriptional regulators such as PGC-la can be regulated, unlike that in ordinary cancer cells, and thus the function of major metabolic regulators may differ from that in ordinary cancer cells. Through this different metabolic regulatory ability and the regulation of cell signaling mechanisms connected thereto, the cancer stem cells acquire resistance to apoptosis under nutrient deprivation and have the ability to invade and/or metastasize. However, the cancer stem cells are not limited thereto, as long as they can differentiate into ordinary cancer cells (including other types of cancer cells). According to one embodiment of the present disclosure, the cancer stem cells may be derived from breast cancer or gastric cancer, but are not limited thereto.

The resistance to the anticancer drugs may mean showing extremely low sensitivity to the anticancer drug treatment, so the cancer that is resistant to the anticancer drug by a treatment method such as chemotherapy may be resistant to a specific anticancer drug from the beginning, or may not show resistance at the beginning but is shown later when it is no longer sensitive to the same therapeutic agent due to genetic mutations in cancer cells after drug treatment for a long time. The resistance to the anticancer drugs may be obtained as cancer cells acquire stem cellularity and become cancer stem cells, but is not limited thereto. In the present disclosure, the anticancer drug is not particularly limited in kind, but may preferably be a drug for treating breast cancer or gastric cancer. Specifically, the anticancer drug may be, but is not limited to, at least one selected from the group consisting of nitrogen mustard, imatinib, oxaliplatin, rituximab, erlotinib, neratinib, lapatinib, gefitinib, vandetanib, nilotinib, semaxanib, bosutinib, axitinib, cediranib, lestaurtinib, trastuzumab, gefitinib, bortezomib, sunitinib, carboplatin, sorafenib, bevacizumab, cisplatin, cetuximab, Viscum album, asparaginase, tretinoin, hydroxycarbamide, dasatinib, estramustine, gemtuzuabozogamicin, ibritumomabtiuxetan, heptaplatin, methyl aminolevulinic acid, amsacrine, alemtuzumab, procarbazine, alprostadil, holmium nitrate chitosan, gemcitabine, doxifluridine, pemetrexed, tegafur, capecitabine, gimeracin, oteracil, azacitidine, methotrexate, uracil, cytarabine, fluorouracil, fludargbine, enocitabine, flutamide, decitabine, capecitabine, mercaptopurine, thioguanine, cladribine, carmofur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, peplomycin, temsirolimus, temozolomide, busulfan, ifosfamide, cyclophosphamide, melphalan, altretamine, dacarbazine, thiotepa, nimustine, chlorambucil, mitolactol, leucovorin, tretinoin, exemestane, aminogluthecimide, anagrelide, navelbine, fadrozole, tamoxifen, toremifene, testolactone, anastrozole, letrozole, vorozole, bicalutamide, lomustine, vorinostat, entinostat, 5FU and carmustine, and preferably, gemcitabine, cisplatin, 5FU, capecitabine, olsaliplatine, vorinistat and Entinostat.

According to one embodiment of the present disclosure, the cancer stem cells may be an undifferentiated cell capable of differentiating into one or more cancer cells selected from the group consisting of breast cancer, gastric cancer, uterine cancer, brain cancer, rectal cancer, colon cancer, lung cancer, skin cancer, blood cancer and liver cancer, and may preferably be at least one of the breast cancer stem cells and gastric cancer stem cells, but is not limited thereto.

The energy metabolic process refers to a series of activities related to the energy production and utilization of living organisms. That is, the metabolic activity includes all of a series of activities that synthesize various metabolites necessary for the activities of living organisms through various biosynthesis, after passing through a digestive process that absorbs energy sources from the outside and converts them into the energy forms that are most readily available to living organisms. At this time, the case where the base product which is the source of energy metabolism is deficient is called metabolic stress, and the case of exposure to metabolic stress for a long time is called chronic metabolic stress.

The chronic glucose-deficient medium refers to a medium from which glucose is removed from a general nutrient medium, and refers to a medium for continuously culturing cancer cells in a medium deficient in glucose. According to one embodiment of the present disclosure, the chronic glucose-deficient medium may include culturing cancer cells in glucose-deficient medium for at least 3 days. Preferably, they may be cultured for at least 5 days and even more preferably for at least 7 days, but is not limited thereto.

The mitochondria, one of the cell organelles, is involved in cell respiration and plays a role in synthesizing ATP, an energy source, through foods brought into the body. Hydrogen ions formed between the inner and outer membranes of the mitochondria flow into the inner membrane of the mitochondria, and the ATP synthase combines phosphoric acid and ADP (a combined form of two phosphoric acid and adenosine) to make ATP (a combined form of three phosphoric acid and adenosine). The mitochondria can multiply themselves. There is a unique DNA in the mitochondria and a unique protein synthesis system. The mitochondria may be present in cancer cells, and the mitochondria in cancer cells are not regular and tend to decrease in number when compared to normal cells, but the change in the centrosome and the Golgi body is not very distinct.

The mitochondrial remodeling means that the mitochondria are reset in terms of structure and function, which includes mitophagy of mitochondria (digestion of damaged mitochondria), cleavage, fusion and biogenesis (mitochondrial production). The cleavage and fusion of mitochondria can be recognized as an essential process for cell survival and can also have an important effect on disease development. In particular, diseases in which mitochondrial remodeling is involved may include cancer, cardiovascular diseases, and neurodegenerative diseases. According to one embodiment of the present disclosure, the mitochondrial remodeling may include a process of fusion and cleavage between the mitochondria. According to another embodiment of the present disclosure, the mitochondrial fusion and cleavage process may be repeated during the process of establishing a cancer stem cell. Preferably, the mitochondrial fusion and cleavage process is repeated in a balanced way, and when fusion is relatively predominant while maintaining the form so that the cancer stem cell is established, the average length of the mitochondria may become longer.

When the mitochondrial fusion occurs, a long structure of the mitochondria is formed, which is caused by the action of three kinds of GTPases (Mfn1, Mfn2, OPal). Mitofusion proteins Mfn1 and Mfn2 are involved in the fusion of the mitochondrial outer membrane, and Opal is involved in the fusion of the mitochondrial inner membrane. Mitochondrial cleavage, on the other hand, is caused by the action of the mitochondrial outer membrane proteins Fis1 (Fission protein 1), Mff (Mitochondrial fission factor), and GTPase Drp1 (Dynamin-related protein 1). Drp1 is usually present in the cytoplasm and flows into the mitochondrial outer membrane when the mitochondria cleaves. Fis1 and Mff may function as adapter proteins for Drp1.

The glucose absorption rate refers to the rate at which the mitochondria absorb, from a medium, glucose, which is one of the energy sources required to produce ATP, which is energy. According to one embodiment of the present disclosure, the cancer stem cells had a lower glucose absorption rate of mitochondria than cancer cells before induction.

The biomarker generally refers to an indicator that can detect changes in the body using proteins, DNA, RNA (ribonucleic acid), and metabolites. In the present disclosure, biomarkers capable of identifying stem cells were used. Specifically, CD44, ESA, SSEA-3, and Oct4 may be biomarkers for stem cell detection, but are not limited thereto.

The cAMP-PKA signal transduction process refers to a signaling system in which cAMP, which is a secondary messenger, activates PKA.

The cAMP is a substance produced in ATP by adenylate cyclase present in the cell membrane, and becomes an intracellular transfer factor of hormonal action. In other words, it is an intermediate that acts as a secondary signal carrier of water-soluble hormones and finally activates PKA and PLC to make cells react to hormones.

The PKA (protein kinase A) is an enzyme that is activated by cAMP. Specifically, when a ligand binds to a G-protein coupled receptor (GPCR), adenylate cyclase (AC) is activated via a G-protein. This enzyme converts ATP into the secondary messenger cAMP, and the generated cAMP activates PKA. The PKA enzyme phosphorylates the serine side chain or threonine side chain of the protein. The cascade activates glycogen synthase, tyrosine hydroxylase, and cAMP responsive element binding protein (CREB). In the present disclosure, cAMP-PKA signal transduction may be activated in cancer stem cells.

The pCREB is one of materials related to the cAMP-PKA signaling process, and refers to phosphorylated CREB. CREB is a cAMP response element-binding transcription factor that binds to specific DNA and regulates transcription of downstream genes. Signals are activated through a signaling system triggered by binding to receptors, and activated CREB binds to the CRE region to excite CBP (CREB binding protein) and regulate the activity of a particular gene.

The PGC-1α is one of the transcription factors that regulate genes involved in an energy metabolic process. In particular, it is a major regulator of mitochondrial biogenesis and can regulate its activity in response to the pCREB. That is, the PGC-la may serve as a direct link between external physiological stimulation and mitochondrial biogenesis.

The oxygen consumption rate (OCR) refers to the rate at which the mitochondria consumes oxygen in the process of generating energy.

A method for producing cancer stem cell according to another aspect of the present disclosure may include: (1) culturing the isolated cancer cells in a nutrient medium; (2) removing the nutrient medium and adding a glucose-deficient nutrient medium; (3) maintaining the culture for at least 3 days in the glucose-deficient medium; and (4) establishing a cancer cell line by repeating a process of obtaining surviving cancer cells after the maintaining step and maintaining them in a glucose-deficient medium again.

The nutrient medium refers to a composition for culturing cells, and according to one embodiment of the present disclosure, the nutrient medium may be DMEM, RPMI 1640, MEM medium. Preferably, it may be RPMI 1640.

According to one embodiment of the present disclosure, the cancer cells may be isolated by pulverizing the cancer tumor tissue obtained from cancer patients and decomposing the matrix to isolate cancer cells. The isolation of the cancer cells may generally include physical and chemical treatment of the general cancer tissue to isolate cancer tissues from patients to obtain cancer cells.

According to one embodiment of the present disclosure, the culture in the glucose-deficient medium may include maintaining for at least 3 days as described above, preferably for at least 5 days, more preferably at least 7 days, but is not limited thereto.

According to one embodiment of the present disclosure, repeating the culture in the glucose-deficient medium may be carried out 7 or more times, preferably 9 or more times, more preferably 11 or more times, but may be repeated until cancer stem cells are established.

According to one embodiment of the present disclosure, the maintaining of the culture may include maintaining until the time when the number of cancer cells falls below 20% of the number of cancer cells before the glucose-deficient nutrient medium is added. This is to establish metabolic stress, that is, cancer cells having the characteristics of the cancer stem cell suitable for the purpose of the present disclosure among cancer cells cultured in a glucose-deficient medium. According to one embodiment of the present disclosure, the maintaining of the culture may include maintaining until the time when the number of cancer cells falls below 10% of the number of cancer cells before the glucose-deficient nutrient medium is added, but is not limited thereto.

The scope of the present disclosure is represented by the following claims, and it should be construed that all changes or modifications derived from the meaning and scope of the claims and their equivalents are included in the scope of the present disclosure.

1. A cancer stem cell derived from cancer cells and induced after being cultured in a chronic glucose-deficient medium, wherein the cancer stem cell has a mitochondrial remodeling.

2. The cancer stem cell of claim 1, wherein the cancer cell is a breast cancer cell.

3. The cancer stem cell of claim 1, wherein the cancer cell is a gastric cancer cell.

4. The cancer stem cell of claim 1, wherein in the mitochondrial remodeling, mitochondrial fusion and fission are repeated, but the fusion is relatively predominant, and an average length of the mitochondria is increased.

5. The cancer stem cell of claim 1,
wherein, in relation to a biomarker, the cancer stem cell shows the following characteristics:
(1) expression of stem cell markers CD44, ESA and SSEA-3 increases more than in parent cells prior to induction, but expression of Oct4 remains unchanged.

6. The cancer stem cell of claim 1, wherein, in relation to mitochondria, the cancer stem cell comprises at least one of the following characteristics:
(1) a glucose absorption rate is lower than in parent cells prior to induction;
(2) a ratio of nucleus DNA (nDNA) to mitochondrial DNA (mtDNA) is higher than in parent cells prior to induction, and mitochondria has a long shape by fusion;
(3) a concentration of cytosolic NAD+/NADH involved in cellular energy metabolism is higher than in parent cells prior to induction,
(4) in cell nucleus and cell substrate, expression of pCREB and PGC-la involved in cAMP-PKA signal transduction is higher than in parent cells prior to induction, respectively; and
(5) an oxygen consumption rate (OCR) increases.

7. The cancer stem cell of claim 1, wherein the cancer stem cell comprises at least one of the following characteristics:
(1) an ability to resist apoptosis in glucose-deficient environment is more competent than in parent cells prior to induction; and
(2) anticancer drug resistance is higher than in parent cells prior to induction.

8. A method for producing cancer stem cells comprising:
culturing isolated cancer cells in a nutrient medium;
removing the nutrient medium and adding a glucose-deficient nutrient medium;
maintaining the culture for at least 3 days in the glucose-deficient medium; and
establishing a cancer cell line by repeating a process of obtaining surviving cancer cells after the maintaining step and maintaining the cancer cells in a glucose-deficient medium again.

9. The method of claim 8, further comprising pulverizing cancer tumor tissues obtained from cancer patients and decomposing matrix to isolate cancer cells before culturing the cancer cells in the nutrient medium.

10. The method of claim 8, wherein the cancer cell is a breast cancer cell.

11. The method of claim 8, wherein the cancer cell is a gastric cancer cell.

12. The method of claim 8, wherein in the maintaining, the culture is maintained until the number of cancer cells falls below 20% of the number of cancer cells before the glucose-deficient nutrient medium is added.

13. The method of claim 8, in the maintaining, the culture is maintained until the time when the number of cancer cells falls below 10% of the number of cancer cells before the glucose-deficient nutrient medium is added.

14. The method of claim 8, wherein the repeating process is performed seven or more times.
